(19) United States
(12) Patent Application Publication
(10) Pub. No.: US 2016/0030148 A1
(43) Pub. Date: Feb. 4, 2016

(54) Implantable Reinforcement Prothesis, In Particular for Reinforcing the Abdominal Wall

(71) Applicant: BIOM'UP, Saint-Priest (FR)

(72) Inventor: Jean-Pierre Cossa, Paris (FR)

(21) Appl. No.: 14/782,897

(22) PCT Filed: Apr. 11, 2014
(86) PCT No.: PCT/EP2014/057452

§ 371 Date: Oct. 7, 2015

(30) Foreign Application Priority Data
Apr. 11, 2013 (FR) 1353277

(51) Int. Cl. A61F 2/00

(52) U.S. Cl. CPC A61F 2/0063; A61F 2002/0068; A61F 2250/0031; A61F 2250/0023

(57) ABSTRACT

The implantable reinforcement prosthesis, in particular for reinforcing the abdominal wall, comprises a reinforcement part (10) forming a netting intended to be incorporated into the inner surface of the wall (1) in order to reinforce said wall and a plurality of flat strips (20; 120) made from textile material extending from the edges of the reinforcement part and intended to be placed in transfixing penetration in the wall to each side of the area to be reinforced. According to a particular provision, the strips (20; 120) comprise anchoring wires (30; 130) intcorporated by interleaving into said strips, and extending along the length of said strips. The strips can be formed from two different segments of strips, depending on the region of the wall in which they are to be positioned.

The present invention relates to the general field of implantable reinforcement prostheses intended to be implanted inside the body of a human being or animal. Such prostheses are intended to ensure the reinforcing of a cell wall (or biological tissue) in which for whatever reason full or partial tears have weakened said wall.

Typically, the anterior abdominal wall is composed of so-called broad muscles organised into different axes and layers to guarantee optimal elasticity for the abdominal muscles. Defects known as hernias are likely to appear in this wall either spontaneously or after surgical procedure requiring operative repair.

For such repair is known to have recourse to a prosthetic reinforcement mesh to seal the deteriorated wall and guarantee long-lasting results, which requires the holding in place of the prosthesis in relation to the wall for sufficient time to ensure healing. Such mesh is similar to a piece of synthetic fabric affixed onto the weakened region much like a puncture patch, on the inner side of the abdominal wall, the mesh pores being intended to become gradually colonised with scar tissue integrating the mesh into the muscular architecture. The shape of this mesh is generally rectangular or oval, the size adapted to the extent of the parietal deficiency to be repaired so that it is covered with a sufficient peripheral bearing surface. Within a few weeks a kind of physiological glue is thus formed, gluing the prosthesis to its application site and guaranteeing definitive parietal reinforcement. To ensure immediate repair of the tear and gradual subsequent reinforcement resulting from anchoring thereof in wall tissues, the prosthesis must be held adequately in position without any risk of being displaced in relation to the wall. For this purpose securing sutures are used passing through the abdominal wall and attached thereto, these not being easy to handle, or biological glues that are still insufficiently reliable, or staples which may or may not be absorbable and form the method most frequently used at the present time.

From FR2914178 an implantable reinforcement prosthesis is known comprising a woven or nonwoven textile reinforcement and anchor sutures joined to said reinforcement and intended to be passed through the surrounding biological tissue of the region to be reinforced, to ensure the securing of the prosthesis, the anchor sutures being provided with conformed anchor barbs so that they can be freely passed through the tissue in one direction and then come to be anchored in the tissue on the periphery of the region covered by the reinforcement part, to form check means of said sutures and thereby firmly hold the prosthesis in place. The sutures shown in the document are similar to the sutures called anchor sutures or barbed sutures known also from EP2092895 for example.

US2011130774 and US20090228021 also describe prostheses comprising a reinforcement provided on its periphery with anchor sutures intended to be inserted in the wall surrounding the region to be repaired covered by said reinforcement.

Anchor sutures are evidently flexible but have some rigidity however due to their cross dimension and to the mechanical properties of their constituent material, typically plastic materials such as polypropylene or polyglactin which subsequently impart some elasticity thereto that is non-controlled however. In other words, when at rest these sutures have a tendency to move in disorderly manner, forming curves and loops as clearly illustrated in the drawings of aforementioned documents FR2914178 and US200090228021.

These properties of anchor sutures may give rise to problems when implanting a said prosthesis, all the more so when it is envisaged to implant the prostheses under laparoscopy.

It is recalled that surgical procedures under laparoscopy are the reproduction of so-called conventional surgical procedures (requiring a large incision) but conducted using a mini-invasive approach without any large opening and hence without direct visual or manual control. The benefit for the patient is a reduction in pain, hospital stay time and recovery time and minimisation of parietal or cosmetic injury. The performing thereof demands technical constraints however and specific equipment.

The implanting of prostheses for hernia repair under laparoscopy is performed using a camera positioned on a kind of periscope 1 cm in diameter connected to a monitor screen and enabling the surgeon to observe the entire operative region without having to make a large incision. The prosthesis is rolled up like a cigarette paper so that it can be inserted via the periscope tube into a chamber created under the abdominal wall to be repaired by insufflation with gas. When the prosthesis is fully inserted in this chamber it is unrolled and placed in position on the region to be repaired.

The particularity of laparoscopic repair of the anterior abdominal wall is that of requiring the positioning of this prosthetic mesh directly inside the abdominal cavity on the peritoneum i.e. the membrane lining the inner side of the abdominal muscles. To ensure the maintained positioning and stabilisation of the prosthesis it is routinely attached using suture or stapling techniques the efficacy of which is not always perfect. It follows that early displacement of the prosthesis occurs before the consolidation time of scar tissue.

The use of prostheses comprising anchor sutures such as aforementioned could facilitate attachment and improve stabilisation. However the above-indicated problems of anchor sutures are particularly crucial when procedure is performed under laparoscopy. The anchor means of the sutures must not hamper insertion of the prosthesis into the abdomen. The sutures are therefore placed folded against the repair portion before this portion is rolled up. The prosthesis is deployed inside the cavity generated in the abdomen. However, on account of their anchor parts, the sutures may remain caught on the fabric of the repair device or conversely may become haphazardly deployed with the risk of becoming caught at non-desired positions on the cavity wall or on the intestinal loops, or they may become knotted with each other. In addition, these sutures arranged in disordered manner may hamper the proper positioning of the repair portion. Also, it may be difficult for the surgeon to take hold of these ill-deployed sutures possibly knotted together so that they can be passed through the abdominal wall and thereby ensure the anchoring thereof and securing of the prosthesis.

It is the objective of the present invention to solve the above-mentioned problems and has the particular objective of allowing the repair of hernias, in particular but not exclusively via laparoscopic route, in simpler and more efficient manner.

It sets out to propose a prosthesis that is particularly capable of ensuring repair via precise positioning of the reinforcement portion and to ensure the reliable, maintained positioning of this portion in relation to the abdominal wall thus guaranteeing rapid integration of the prosthesis in the tissues and hence for rapid, sustained repair.

The invention particularly sets out to propose a prosthesis specifically adapted for implantation via laparoscopic surgery under optimal conditions of operability and rapidity.

With these objectives in view, the subject of the invention is an implantable reinforcement prosthesis to reinforce a biological wall of the human body and in particular the abdominal wall, comprising a reinforcement portion typically in the form of woven or nonwoven mesh fabric intended to be integrated on the inner surface of the wall for the reinforcement thereof.

According to the invention, the prosthesis is characterized in that it comprises a plurality of flat strips called anchor strips in textile material extending from the edges of the reinforcement portion. These strips are intended to be positioned via transfixing penetration into the wall either side of the region to be reinforced.

The prosthesis of the invention is more particularly described with reference to the appended claims.

The anchor strips are intended to be positioned, like the securing sutures of prior prostheses described above, by transfixing penetration into the muscular wall either side of the region to be repaired or reinforced. The strips therefore immediately tightly tie the prosthesis onto the implantation site by means of peripheral transfixing penetrations in the muscular wall. The natural colonising fibrosis of the trans-parietal strips, which is all the more extensive as the strips have a relatively large width and hence surface area for development of said colonisation, very rapidly allows sufficiently robust anchoring to be obtained to withstand the forces exerted in everyday life thereby allowing optimal integration of the prosthetic mesh stabilised and secured around the region of parietal deficiency to be reinforced. When this mesh is finally stabilised a kind of physiological glue is formed, gluing the prosthesis onto its insertion site.

As indicated above, one of the major problems is to ensure the most reliable stability possible, both initially after implantation and over the longer term. With maturing of the peri-prosthetic fibrosis process which is generated and sought after as principle of repair, secondary contraction phenomena of the mesh can nevertheless be observed under the retractile effect of the conjunctive scar tissue, similar to a skin scar which can progressively collapse and even disappear over the years. The prosthetic cover surface can then be considerably reduced, which may lead to late recurrence of the hernia. The prior art anchor sutures effectively ensure immediate maintaining of the prosthesis at the time of insertion, but they do not allow efficient maintained positioning over time and therefore do not make provision for the effects of these subsequent contractions of the prosthetic mesh. In comparison with anchor sutures and advantageously, the strips of the prosthesis of the invention, through their increased contact surface between the strips and the muscular tissues of the abdominal wall, allow improved maintained tensioning of the prosthesis via the natural colonising fibrosis occurring on these strips passed through the abdominal wall, when compared with known securing sutures or anchor sutures. It will incidentally be noted that this maintaining will be improved if the trans-parietal passing of the strips is performed obliquely which will provide a larger contact surface between the strips and the tissues of the abdominal wall than if this passing is performed perpendicular to the wall.

A further advantage of these strips is that since they are not thread-like but flat they display better global rigidity than sutures, facilitating the grasping thereof in the abdominal cavity so that they can be passed through the wall.

Preferably, the free end of the strips is profiled to facilitate insertion thereof through the incision formed in the wall. It is possible for example to provide for a pointed free end.

Preferably, the strips are completed with anchor sutures 30 integrated via interleaving in said strips and extending along the length of said strips. These anchor sutures may also have an antibacterial function.

By interleaving it can particularly be understood that said sutures can be threaded in the constituent fabric of the strips after the manufacture of this fabric, or optionally cut out together with the strips which would simplify manufacture of the fabric but would require a subsequent operation to thread in these sutures. Said sutures can also be directly woven during manufacture of said constituent fabric of the strips.

The use of antibacterial sutures e.g. of Vicryl PLUS® type allows the providing of additional anti-infection prophylaxis in response to the risk of prosthesis contamination via exogenous per-cutaneous route.

The anchor sutures also called self-locking or barbed sutures e.g. of V-Lock® type or the like have raised portions for example in the form of barbs oriented obliquely in relation to the longitudinal direction of the suture and allowing the initial maintaining of the prosthesis to be completed and improved via the immediate anchoring they provide in the biological tissues, muscular tissues in particular, when passed through the abdominal wall.

One major advantage of the invention is that, after the prosthesis has been inserted in the abdominal cavity but before the strips are inserted in the abdominal wall, the flat shape and structure, in particular woven structure, of the strips ensures rigidifying of the anchor sutures optionally joined to said strips, or at least prevents the disorderly deployment of these sutures as described for the prior art, which largely facilitates grasping of the sutures and strips for insertion thereof in the wall. This advantage is of particular interest for laparoscopic surgery since it offers greater handling ergonomics than observed with the sutures of the aforementioned prior art prostheses.

According to one preferred provision, the strips are formed in a single piece with the reinforcement portion e.g. being simultaneously cut out with the reinforcement portion from a sheet of a constituent material of said reinforcement part. This embodiment is particularly simple and allows limiting of the manufacturing costs of the prostheses. Also the inserting of the anchor sutures in the strips can optionally also be performed simply by type of weaving of these sutures in the longitudinal direction of the strips in the woven material with wide weft forming the prosthesis and said strips.

The strips preferably have a length in the order of 10 cm to 15 cm and width of 5 mm to 10 mm. The reduced width allows limiting of the incision required to pass the strips through the abdominal wall, it being understood however that the strips must be sufficiently wide first to ensure sufficient maintaining of the anchor sutures and secondly to provide a sufficient surface area for their joining to the wall via the natural fibrosis that will develop on the contacting thereof as previously indicated.

According to one embodiment which can be taken alone or in combination with the aforementioned characteristics, each strip comprises two different longitudinal segments, namely:
A first segment—called anchor segment—essentially intended to be in contact with the muscular tissue of the patient's wall; and
a second segment—called grasping segment—essentially intended to be in contact with the cutaneous and sub-cutaneous tissues of the wall.

Preferably the two segments are each portions of strip.

Further preferably the strip comprises solely these two strip segments, and does not for example comprise any additional suture for grasping of the assembly.

Each strip irrespective of shape may comprise one or more anchor sutures and preferably comprises at least two thereof integrated for example close to the edges of the strips. According to one preferred embodiment, each strip comprises three or four anchor sutures extending in the direction of the strip and regularly spaced apart over the width of said strip.

The reinforcement portion and the strips are essentially made of polypropylene, polyurethane and/or polyester.

The anchor sutures are preferably made in absorbable monofilament.

Also, the anchor sutures preferably have a self-locking architecture integrating one-way members in particular.

The prosthesis is typically formed of a synthetic fabric in polypropylene, polyurethane and/or polyester, ideally with high porosity i.e. with sufficiently large mesh size (pore diameter of 1 mm to a few millimetres, preferably larger than 3 millimetres) intended to facilitate colonisation by scar tissue to repair and consolidate the wall.

At the reinforcement this mesh is preferably covered on one side with an anti-adherent film intended to be placed facing inwardly into the abdominal cavity, the objective of which is to obtain the prevention of intestinal adhesions on the prosthesis. Said anti-adherent film may comprise at least one absorbable biological macromolecule for example and/or at least one synthetic and/or natural polymer such as collagen. For example a reinforcement similar to the COVMESH™ product marketed by BIOM'UP can be used.

To facilitate the integration of the strips in the tissues, as will be seen below, this anti-adherent film may optionally be omitted on the strips or it may not be formed thereupon.

With the prosthesis in the state ready to be implanted, the free ends of the strips are held joined together by a restraining tie or absorbable dot of glue, and the prosthesis is rolled up with the strips inside the roll. Therefore when the prosthesis is inserted in the abdominal cavity the strips and anchor sutures are properly held within the rolled-up prosthesis and there is no risk that they will catch on the trocar used or on neighbouring tissues.

Once placed in the abdominal cavity, the strips are released and are again able to extend over the plane of the reinforcement portion whilst ensuring holding of the anchor sutures to allow easier grasping of the strips and sutures for insertion thereof in the incisions provided for this purpose in the abdominal wall.

Preferably the free end of each strip comprises visual marking i.e. marking enabling the surgeon to differentiate the free end of the strip from the remainder of the strip and preferably from the remainder of the prosthesis. This marking facilitates the positioning of the prosthesis since it allows the surgeon—operating within a confined medium—to rapidly take hold of the end of the strip to deploy and insert the strip through the parietal incision.

The reinforcement portion may be of rectangular shape (square in particular), the strips then extending from the corners of the reinforcement respectively, or of oval shape (circular in particular) with the strips respectively extending along the long axis and short axis of the oval. Other shapes of the reinforcement could be used and the number and arrangement of the strips adapted accordingly, maintaining the objective of ensuring the best possible holding in position of the prosthesis and the best immobilisation thereof against the peritoneum, in keeping with the site of implantation and required size.

Other characteristics and advantages will become apparent from the following description of a prosthesis conforming to the invention and use thereof to reinforce a herniated abdominal wall.

Reference is made to the appended drawings in which:

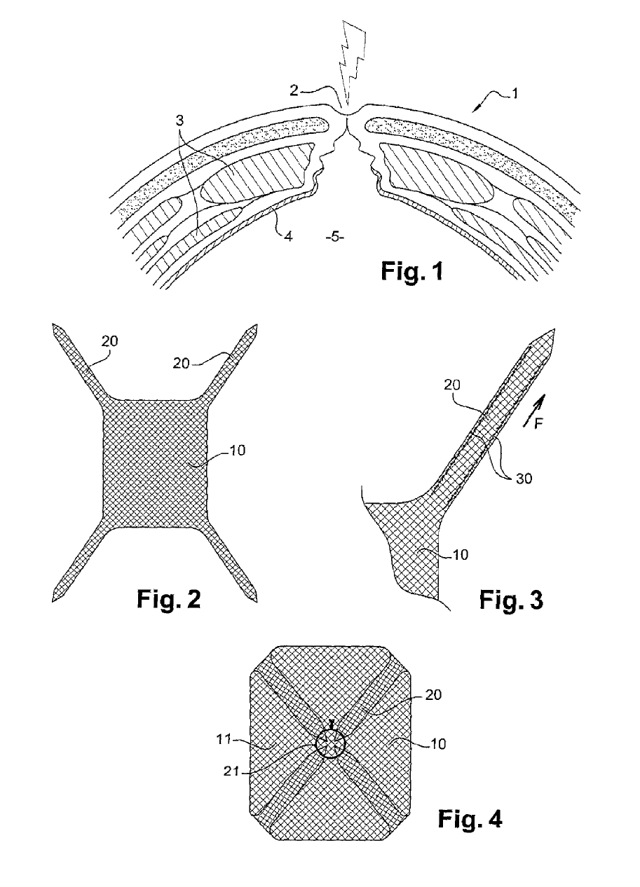

FIG. 1 is a cross-sectional view illustrating a herniated abdominal wall;

FIG. 2 is a plan view of a prosthesis according to the invention;

FIG. 3 is a detailed view of a strip of the prosthesis;

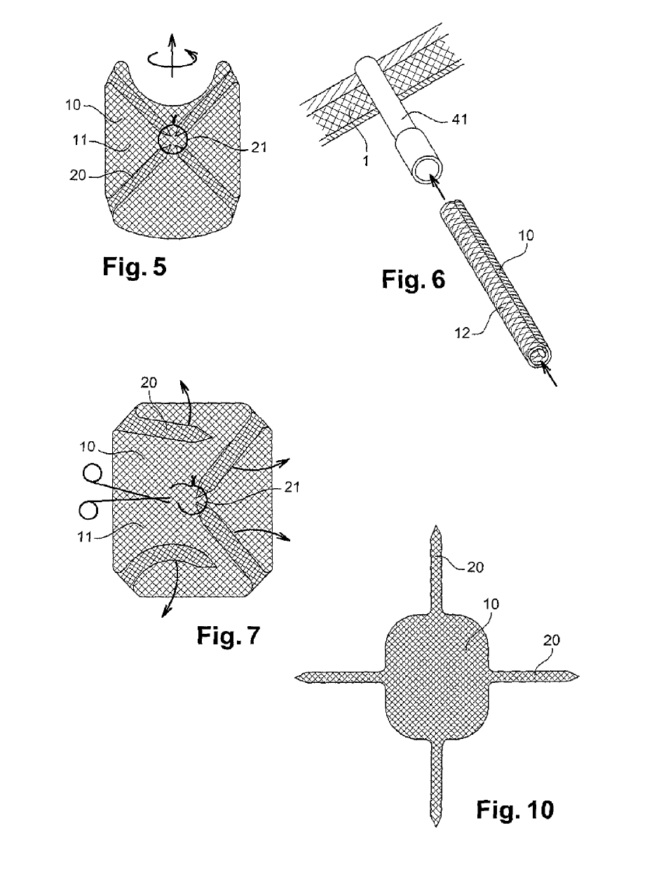

FIGS. 4 and 5 show the preparation of the prosthesis for its insertion into the abdominal cavity;

FIG. 6 schematically illustrates this insertion with the prosthesis being rolled-up;

FIG. 7 shows the separation of the strips and their expansion after the prosthesis has been inserted into the abdominal cavity;

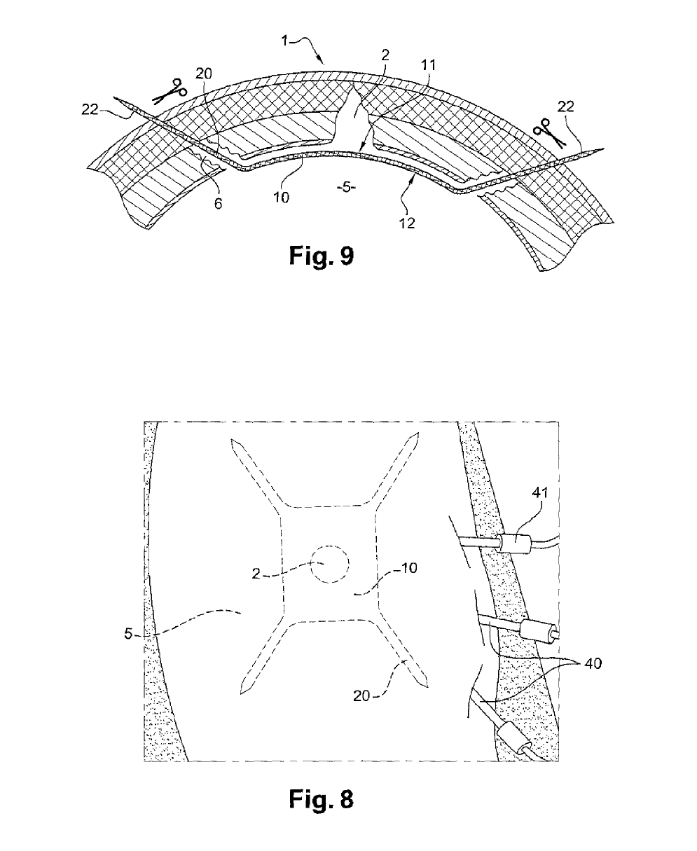

FIG. 8 is a plan view illustrating the prosthesis in position, centred on the region to be repaired, before passing the strips through the abdominal wall, and the instrumentation required for the procedure;

FIG. 9 is a cross-section schematically illustrating the prosthesis in place against the peritoneum, and the ends of the strips projecting outwardly ready to be cut;

FIG. 10 gives a variant of embodiment of the prosthesis;

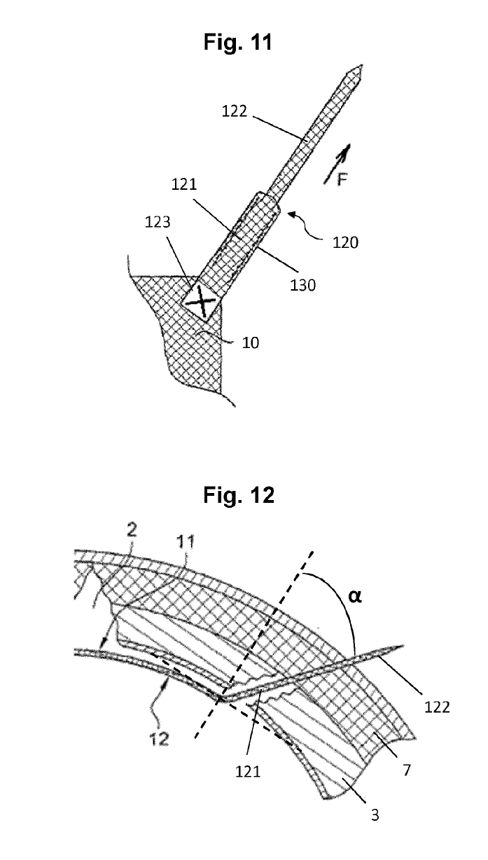

FIG. 11 is detailed view of a variant of embodiment of a strip of the prosthesis;

FIG. 12 illustrates the oblique insertion of a strip in FIG. 11 through a patient's abdominal wall.

FIG. 1 is a cross-section illustrating the abdominal wall 1 with the hernia 2 showing in particular the flat muscles 3 and peritoneum 4 against which the prosthesis is to be secured to repair the hernia tear and reinforce the entire wall until complete healing and reconstitution of the wall in its normal state.

The proposed prosthesis illustrated in FIG. 2 comprises a reinforcement portion 10 of general rectangular shape for example and intended to ensure this repair by application against the peritoneum and progressive integration into the tissues, and several strips e.g. four strips 20 respectively extending from the apexes of the reinforcement portion 10.

Preferably the strips 20 extend outwardly from the reinforcement portion 10 along one same plane.

As illustrated in FIG. 3, the strips preferably have a profiled end, having a pointed profile for example, to facilitate insertion through the incision made in the patient's abdominal wall.

The dimensions of the reinforcement portion are determined in relation to the hernia to be repaired and may typically be in the order of 15 cm×10 cm or 20 cm×15 cm or even larger.

The length of the strip is generally dependent upon the size of the prosthesis whilst taking into account the minimum length required for passing through the entire wall in an oblique direction. It is also preferable that the length should be sufficient to allow temporary holding together of the different strips in folded position without the folded strips projecting from the reinforcement portion. Therefore for a reinforcement of rectangular shape having a diagonal of length D, provision is preferably made for strips having a length equal to or longer than one half of the diagonal D, and preferably shorter than the length D of the diagonal.

The length of the strips is 10 cm to 15 cm for example, with a width in the order of 5 mm to 10 mm.

The strips are preferably formed in a single piece with the reinforcement portion, for example by cutting out the shape illustrated in FIG. 2 from an adapted sheet of fabric.

However the strips may also be independent parts added on and attached to the periphery of the reinforcement portion. For example, the strips can be joined to the reinforcement portion by over-moulding, welding, knotting and/or gluing. In this case provision must be made so that part of the strip is intended to be covered by the reinforcement portion at the point of attachment, and cannot therefore be used as transfixing portion. The length of the strip is therefore adapted to the effective desired length, the effective length corresponding to the length of the strip portion intended to be passed through the wall.

The reinforcement portion preferably has two sides having different specific properties.

Preferably, for use in the abdominal cavity, the prosthesis used is a so-called two-sided prosthesis i.e. its two sides are different:
a so-called outer side 11 intended to be applied against the abdominal wall and having adhesive properties;
a so-called inner side 12 having anti-adherent properties.

The desired characteristic of the outer side 11 is the most efficient and rapid adhesiveness possible, to ensure the fastest, most reliable anchoring possible of the prosthesis onto the chosen site. For example, a piece of synthetic fabric can be used in polypropylene, polyurethane and/or polyester in the form of a woven or knitted mesh having a weft or mesh size in the order of 1 mm to a few millimetres.

The adhesion generated by gradual tissue in-growth into the prosthesis is never immediate and the prosthesis must therefore be immediately held firmly in position using different means, as already indicated. Scar tissue around the prosthetic mesh pores then takes over ensuring final integration thereof which alone can guarantee the reliability of repair.

The inner side 12 is formed so as to be the least adhesiogenic possible since it is intended to lie opposite the inner side of the abdominal cavity facing the content thereof i.e. opposite the intestinal loops which are mobile and hence likely to be in contact with the prosthesis, especially after collapse of distension of the abdominal cavity when pneumoperitoneum insufflation is stopped at the end of procedure, as will be described below. For this purpose the inner side 12 of the prosthesis is formed of an anti-adherent film for example covering one side of the fabric piece and intended to obtain the prevention of intestinal adhesions on the prosthesis in the most efficient way possible.

This anti-adherent film is typically formed of a cover layer attached onto the constituent fabric of the outer side, in manner known per se. For example, the anti-adherent film is in collagen.

An example of a said reinforcement is the prosthetic fabric COVAMESH™ marketed by BIOM'UP.

More specifically, the reinforcement portion having a fabric part integrating a collagen layer can be designed in accordance with the teachings of international application WO2010125086 or international application WO2011079976.

As can be seen in FIG. 3, the strips 20 may comprise anchor sutures 30, typically of V-Lock® type inserted along the length of the strips and preferably arranged on the edges thereof.

These anchor sutures 30 are positioned by weaving into the weft threads forming the strips 20.

The anchor barbs of these anchor sutures 30 are oriented in the direction indicated by the arrow F allowing sliding within the biological tissues subsequently crossed by the strip carrying said sutures, and ensuring the anchoring in said tissues in the other direction. These anchor barbs therefore act as one-way members with respect to the biological tissues through which they are passed.

The anchor sutures 30 are therefore held by the strips in which they are inserted for as long as these are not inserted into the abdominal wall, and subsequently ensure their anchoring effect in the biological tissues on each side of said strip since said sutures and their barbs are alternately apparent on each of said sides.

Before use, each prosthesis is packaged to allow its insertion into the abdominal wall, for example using a trocar 41 as illustrated in FIG. 6. For this purpose, the strips 20 are folded against the reinforcement portion on its outer side 11 and joined together by a tie suture 21 for example as illustrated in FIG. 4.

The prosthesis is then rolled up as illustrated in FIG. 5 along its longer axis with the strips on the inside of the roll to form a cylinder such as illustrated in FIG. 6 of sufficiently narrow diameter to allow passing into the trocar 41.

In operating mode under laparoscopy, the prosthesis is placed in position in the chamber formed by the abdominal cavity 5 previously distended by insufflation of gas (pneumoperitoneum). The objective of this insufflation is to provide sufficient operating space, general anaesthesia being necessary to allow muscle relaxation and thereby allow distension of the abdominal muscles.

By means of several small elective incisions allowing the positioning of perforating tubes 40 provided with sealed valves (called trocars) as illustrated in FIG. 8, the following are successively placed in position for performing of the procedure:
an optical tube 10 mm in diameter coupled to a high definition camera linked to a monitor screen, to ensure investigation and visual control of the surgical procedure,
different instruments in general having a diameter of 5 mm for dissection, coagulation, ligaturing and other surgical steps.

For procedure to repair the anterior abdominal wall, the technique whereby a region of the wall surrounding the tear is covered on the inside with the prosthesis, the incisions for the camera and instruments are made on one of the sides of the abdominal cavity, away from the region to be repaired as shown in FIG. 8.

The prosthesis being packaged in the form of a cylinder as indicated previously, this cylinder is inserted inside the distended abdominal cavity through a trocar 41 of 10 mm or 12 mm as illustrated FIG. 6. It will be noted that the ease of insertion of the prosthesis allows minimisation of its exposure time to air and digital handling.

Once inserted, the prosthesis tends to unroll naturally via elasticity, this unrolling optionally being assisted and controlled by the instruments handled by the surgeon. The surgeon then cuts the tie 21 holding together the strips allowing the deployment thereof. This deployment is aided first by the volume formed in the abdominal cavity by the distension thereof, and secondly by the natural tendency of the prosthesis to return to a flat shape resulting from its structure and the properties of its constituent material, thereby extending the strips and allowing facilitated subsequent grasping thereof.

The surgeon assists this deployment and centres the prosthesis on the deficiency 2 to be repaired, as illustrated in FIG. 8, bringing the reinforcement portion 10 against the inner side of the abdominal wall.

It will be noted that the facilitated handling of the strips compared with known sutures, through the additional rigidity they provide, means that the risk of undue and potentially traumatic catching of the anchor suture on the nearby intestinal loops may no longer be feared during movements to place the prosthesis in position.

Through the incisions 6 made in the abdominal wall, the strips 10 are successively grasped by mini-forceps and drawn outwardly. As can be clearly seen in FIG. 9, the incisions are preferably made obliquely in the abdominal wall to provide an oblique pathway for the strips 20, thereby ensuring an increased contact surface between each segment of trans-parietal strip and the different muscle layers of the abdominal wall through which they are passed. The superior rigidity of the strips 10 compared with prior art anchor sutures facilitates the handling thereof which can be achieved using a single pair of forceps.

The tensioning of the four strips 10 will allow the centring and optimal flattening of the prosthesis against the abdominal wall, and will provide immediate stabilisation of its seating which will then allow additional intra-abdominal securing by stapling or gluing under excellent conditions of comfort and reliability. By avoiding phenomena of shifting of the prosthesis on the parietal peritoneum, prosthetic stapling is facilitated and the maintained tensioning of the prosthesis obtained by the strips makes it possible in particular to prevent risks of pleating of the prosthesis at the time of stapling.

On completion of the procedure, after releasing the pressure inside the abdominal cavity, the strip excesses 22 are sectioned flush with the plane of the skin as illustrated in FIG. 9 to allow easy burying under a simple skin covering suture.

Via the induced inflammatory reaction to a foreign body generated by the prosthetic fabric, each strip in its parietal tissue environment through which it is passed will progressively become ingrown with a three-dimensional network of collagen fibres ensuring colonisation thereof by conjunctive scar tissue. This secondary anchoring will therefore build up reinforced prosthesis securing, providing against the risk of secondary prosthetic luxation or shrinkage of the prosthetic cover surface under the effect of secondary contraction leading in both cases to phenomena of late recurrence.

When using this type of prosthesis, it is therefore possible to obtain ideal repair of tears in the anterior abdominal wall using a mini-invasive parietal approach, with the use however of large prostheses the size of which is not limited by the incision for insertion since they are inserted rolled up and are seated well away from the region to be reinforced, thereby ensuring optimal distribution of the bearing of the prosthesis against the abdominal wall, more particularly during intra-abdominal pressure increases occurring naturally in everyday life (efforts made for coughing, defecation, vomiting, lifting, etc. . . . ).

Preferably the positioning of the prosthesis is facilitated by the use of a positioning template to locate the points of incision through which the strips are intended to be positioned.

Said positioning template could be supplied with the strip prosthesis in a kit, the template possibly being drawn for example on the sterile packing in which the prosthesis with strips is packaged.

The markings determining the position of the points of incision to be formed are identified before insufflation and hence distension of the abdominal cavity, to allow optimal centring of the prosthesis taking into account the parameters of obliqueness for the trans-parietal pathway of the strips allowing optimisation of their contact surface and anchoring capability, in particular in the trans-muscular layer. Also, it is preferable to estimate the desired margins of the surface covered by the prosthesis in relation to the specific characteristics of the parietal deficiency to be treated (size and direction of the main axis, suture for approximation or not of aponeurotic edges, quality of bearing tissue, anchoring possibilities, parietal adiposity and desired overlapping).

Preferably the positioning template is designed so that the incision inlet points in the patient's skin are placed at a distance allowing an angle of obliquity a to be formed of at least 45°, and preferably an angle of obliquity a of 60°. The term angle of obliquity a used here is the angle formed between a strip and the perpendicular to the plane tangent to the surface of the prosthetic fabric at the point of attachment of the strip, as illustrated in FIG. 12.

For example the positioning template can be formed of a main part having the same shape and the same dimensions as the reinforcement portion of the prosthesis, where ear-shaped elements are arranged in similar manner to the strips, these ears comprising reference points to identify the points of incision to be formed. It can be envisaged that each ear comprises several reference points in relation to the chosen angle of obliquity and/or in relation to the thickness of the wall to be passed through so that the strips re-emerge outside the patient.

According to one particular embodiment illustrated in FIG. 11, the strips 120 associated with the reinforcement portion 10 comprise two different longitudinal segments, namely:
a first segment—called anchor segment 121—intended to be in contact with the muscle layer 3 of the patient's abdominal wall; and
a second segment—called grasping segment 122—intended to be in contact with the cutaneous and subcutaneous layers 7 of the patient's wall extending as far as outside the patient.

The anchor segment 121 of the strip comprises one end joined to the reinforcement portion whilst its opposite end is joined to one end of the grasping segment 122. The second end of the grasping segment corresponds to the free end of the strip intended to lie outside the patient once the prosthesis has been placed in position.

For the strips 120 to be tensioned, they need to be passed through the entire wall and to emerge outside the patient. The effective anchor channel through the wall is the dense, tonic muscle layer in which the strip can be anchored by locking for example and where cell colonisation occurs allowing reinforced adhesion. On the contrary, in the layers nearer the surface in particular the layer of subcutaneous fatty tissue that is more friable, the primary anchoring of the strip is less efficient, the subsequent cell colonisation of the fabric forming the strip also being less reliable in this region. In addition, the presence of a portion of strip in this surface region, fatty region in particular, may generate discomfort for the patient together with a risk of septic contamination of cutaneous origin. It is therefore most advantageous to provide a strip having two separate segments, the first segment being specially sized to promote effective anchoring in the muscle region of the wall, whilst the second segment is sized to minimise its negative impact on the patient whilst allowing tensioning of the strip from outside the patient.

It is to be noted that each of the two longitudinal segments forming the strip are themselves portions of strips in particular having a certain width and having the advantages described above particularly with regard to handling as compared with sutures for example.

To facilitate primary anchoring of the strip in the muscle region 3, the anchor segment 121 may comprise specific anchoring means in the form of anchor sutures 130 for example which may be similar to those previously described. These anchor sutures take part in reinforcing the rigidity of the anchor segment.

Sutures can also be provided in the grasping segment 122 to rigidify the corresponding strip segment.

Preferably this suture does not have specific anchoring means unlike in the anchor segment. In addition, it is desirable that these sutures should have an antibacterial function to reduce the risks of propagation of any infections.

It could also be envisaged that only one and the same suture passes through the two segments of the strip, in which case preferably a complex suture is used having a portion with anchor elements for the anchor segment and a portion devoid of anchor elements for the grasping segment.

Preferably the anchor segment has a width at least twice the width of the grasping segment. The anchor segment may have a width for example of between 5 mm and 10 mm, whilst the grasping segment will have a narrower width in the order of 2 mm to 3 mm. On this account the impact of the grasping segment on the patient will be reduced.

The total length of the strip is substantially the same for an arrangement having several segments as for an arrangement having a single segment as described above. The length of the grasping segment is at least equal however to the length of the anchor segment. According to one preferred embodiment, the grasping segment is at least twice longer than the anchor segment, and preferably at least three times longer.

For example, for a strip having a length in the order of 9-10 cm, an anchor segment can be provided having a length of 20 mm to 30 mm, whilst the grasping segment has a length of 60 mm to 90 mm.

Alternatively or in addition to the sizing and additional anchor means presented above, provision may also be made to form the anchor segment using a different material and weave/knit to those of the grasping segment.

For example it is preferable that the mesh size of the anchor segment should have large pores, with a diameter of one millimetre to several millimetres, preferably 3 mm to 6 mm, to promote cell colonisation whilst allowing the maintaining of a certain porosity (e.g. 60%) after integration of the strips. The suture used is non-absorbable, preferably multi-filament.

The meshing of the fabric forming the grasping segment may be identical to that forming the anchor segment. A grasping segment can also be envisaged having a tighter mesh than the mesh of the anchor segment i.e. having pores of smaller diameter, which will allow reinforcing of the rigidity of this portion of strip without having recourse to other specific means.

The suture used to form the grasping segment may be non-absorbable but it is preferably absorbable, so as to eliminate any discomfort for the patient after integration. In addition, the suture used is preferably mono-filament, to reduce risks of chronic inflammation by bacterial colonisation.

The grasping segment is generally designed to reduce the surface of foreign body implanted in these cutaneous and subcutaneous layers as much as possible, whilst maintaining minimum rigidity of the strip to guarantee easy handling.

Provision can also be made to use antibacterial suture at the grasping segment either in combination with conventional synthetic sutures or exclusively for the weaving/knitting of the portion of strip forming the grasping segment. This will provide additional anti-infectious prophylaxis in response to the risk of prosthetic contamination via exogenous percutaneous route.

The embodiment illustrated in FIG. 11 shows a strip that is independent of the reinforcement portion, and joined thereto at an attachment point 123 located in the corner of the reinforcement portion.

Whether formed of one or several segments, it is preferable that the strips should have some axial elasticity i.e. the capability of elongating along the length of the strip and of returning to their initial length without deformation.

Said axial elasticity guarantees a damping role for the portions of strip in the trans-muscular region, thereby imparting a “tensioner” function to the reinforcement portion, but also allows a certain amount of spontaneous retraction for the segments in the intra-abdominal fat region after drawing the outer protruding part for easier burying thereof when it has been cut flush with the skin.

It is possible for example to use strips having an axial elasticity of 20% to 30%.

The transverse elasticity of the strips may be in the order of 15%.

The strips of the prostheses provided for reinforcing the abdominal wall are preferably designed to meet intra-peritoneal physiological variations in pressure (maximum threshold measured using the Laplace model with 252 cm of water) requiring the maintaining by the prosthetic device of the elasticity of the region of repaired abdominal wall, the recommended thresholds being 25% for the cranio-caudal axis and 15% for the transverse axis.

As mentioned above, the reinforcement portion is preferably two-sided, one of the sides being formed by a fabric mesh and the other side being a layer having antiadhesive properties.

In this case, it is nevertheless preferable that the strips should not be coated with a said anti-adhesive layer since on the contrary good anchoring is desired in particular at the muscle region.

The invention is not limited to the above-described embodiments given solely as examples

In particular the shape of the reinforcement portion may differ from that of a rectangle and may be of oval shape as illustrated in FIG. 10 in which case the strips preferably extend along the axes of this oval, or any other shape adapted to needs. It is to be noted that a specific rectangular shape of the prosthesis may be a square whilst a specific oval shape of the prosthesis may be a circle.

The number of strips may also be modified and the integration of self-locking or antibacterial sutures therein can be obtained when manufacturing the strips by weaving.

Finally, the application of the prosthesis is not limited to laparoscopic use. It will also be noted that the prosthesis is preferably intended for repair of the abdominal wall but can also be used, with various dimensions, so that the same principle of contention can be applied to contain any loss of body substance when direct repair via conventional routes such as direct suturing for example cannot be performed but when temporary or definitive contention must be ensured (e.g. widening plasty, etc.) or when the reconstruction must be planned of loss of organic substance.

BIBLIOGRAPHICAL REFERENCES

| | | |
|---|---|---|
| FR2914178 | EP2092895 | US2011130774 |
| US20090228021 | WO2010125086 | WO2011079976 |

1. An implantable prosthesis to reinforce a wall, in particular the abdominal wall, comprising a reinforcement portion forming a mesh intended to be integrated on the inner surface of the wall to reinforce said wall, characterized in that it comprises a plurality of flat strips in fabric material extending from the edges of the reinforcement portion and intended to be positioned by transfixing penetration in the wall either side of the region to be reinforced, wherein each strip comprises two different longitudinal strip segments, namely:
a first segment called anchor segment—intended to be mainly in contact with the muscle tissues of the wall; and
a second segment called grasping segment—intended to be mainly in contact with the cutaneous and subcutaneous tissues of the wall.

2. The prosthesis according to claim 1, characterized in that the first segment has a width at least twice the width of the second segment.

3. The prosthesis according to claims 1, characterized in that the first segment has a length at least equal to the length of the second segment, preferably a length at least twice the length of the second segment, and further preferably a length at least three times longer than the length of the second segment.

4. The prosthesis according to claim 1, characterized in that the first segment is formed with non-absorbable suture and the second segment is formed with absorbable suture.

5. The prosthesis according to claim 1, characterized in that the first segment and the second segment are pieces of fabric formed by weaving or knitting having a first mesh size and second mesh size respectively, wherein the porosity formed by the first mesh is larger than the porosity formed by the second mesh.

6. The prosthesis according to claim 1, characterized in that the strips comprise anchor sutures integrated by interleaving in said strips, and extending along the length of said strips.

7. The prosthesis according to claim 6, characterized in that the anchor sutures are solely arranged in the anchor segment of each strip.

8. The prosthesis according to claim 6, characterized in that each strip comprises at least two anchor sutures.

9. The prosthesis according to claim 6, characterized in that the anchor sutures are integrated close to the edges of the strips.

10. The prosthesis according to claim 6, characterized in that the anchor sutures are absorbable monofilament sutures.

11. The prosthesis according to claim 6, characterized in that the anchor sutures comprise self-locking one-way members.

12. The prosthesis according to claim 1, characterized in that the reinforcement portion comprises an anti-adherent film on one side, preferably in collagen.

13. The prosthesis according to claim 1, characterized in that in its ready-to-use state the free ends of the strips are held together in particular by a thread and the prosthesis is rolled up with the strips inside the roll.

14. The prosthesis according to claim 1, characterized in that the reinforcement portion has a rectangular shape and the strips extend respectively from the corners of the reinforcement portion.

15. The prosthesis according to claim 1, characterized in that the reinforcement portion is of oval shape and the strips extend respectively along the long axis and the short axis of the oval.

16. The prosthesis according to claim 1, characterized in that the strips comprise antibacterial sutures integrated by interleaving in said strips, and extending along the length of said strips.

17. The prosthesis according to claim 1, characterized in that each strip comprises a free end having specific visual marking intended to differentiate said free end from the remainder of the strip.

18. A kit comprising an implantable prosthesis to reinforce a wall, in particular the abdominal wall, comprising a reinforcement portion forming a mesh intended to be integrated on the inner surface of the wall to reinforce said wall, characterized in that it comprises a plurality of flat strips in fabric material extending from the edges of the reinforcement portion and intended to be positioned by transfixing penetration in the wall either side of the region to be reinforced, wherein each strip comprises two different longitudinal strip segments, namely:
a first segment—called anchor segment—intended to be mainly in contact with the muscle tissues of the wall; and
a second segment—called grasping segment—intended to be mainly in contact with the cutaneous and subcutaneous tissues of the wall;
wherein the kit further comprises a positioning template comprising locating elements to identify inlet points facilitating the forming of transcutaneous incisions intended for transfixing penetration of the strips of the prosthesis.

19. The kit according to claim 18, characterized in that the positioning template is formed on a pack in which the prosthesis is packaged.

20. The kit according to claim 18, characterized in that the positioning template has a main body having a shape substantially identical to the shape of the reinforcement portion, the locating elements being arranged on the main body in the same arrangement as the strips relative to the reinforcement portion, said locating elements being sized for marking of the inlet points at a distance from the corresponding main body at an angle of obliquity α of at least 45° and preferably an angle of obliquity α of 60°.
